(19) United States
(12) Patent Application Publication
(10) Pub. No.: US 2011/0054631 A1
(43) Pub. Date: Mar. 3, 2011

(54) CARTILAGE RESURFACING IMPLANT

(76) Inventors: Yves-Alain Ratron, Grenoble (FR); Michel Hassler, Saint Ismier (FR)

(21) Appl. No.: 12/933,872

(22) PCT Filed: Mar. 21, 2009
(86) PCT No.: PCT/EP09/53381

§ 371 Date: Oct. 26, 2010

Related U.S. Application Data
(60) Provisional application No. 61/047,349, filed on Apr. 23, 2008.

(30) Foreign Application Priority Data
Mar. 21, 2008 (FR) 0851872

(51) Int. Cl. A61F 2/36

(52) U.S. Cl. 623/23.12

(57) ABSTRACT

The invention relates to a cartilage resurfacing implant for replacing all or some of the articular cartilage, characterised in that it is made of a biocompatible material (1) of which the modulus of elasticity is between 20 and 25 GPa, said implant matching the shape of the articular bone end on which it is arranged and comprising at least one aperture (2) enabling at least one plug (3) to be arranged and fixed in place. The invention also relates to a cartilage resurfacing kit comprising at least one implant and at least one plug (3) for treating cartilage lesions.

The present invention relates to the field of arthroplasty and, more specifically, to the methods used to repair articular cartilage.

Articular cartilage is subjected to stresses and lesions owing to its location both in young patients, for example brought on as a result of intense sports activity, or in old patients, for example as a result of a disease or condition such as osteoarthritis. The cartilage may be damaged and, since it cannot effectively repair itself, this damage may lead to the bone itself being directly exposed in the regions where the joints rub against one another, resulting in discomfort which may culminate in immobilisation, pain and deterioration to some extent.

Despite advances within the field of materials and innovations in tissue repair, no satisfactory treatment or material able to mimic the specific properties of articular cartilage in a genuinely satisfactory manner exists. Most of the current methods involve inserting a repair tissue which is basically made of fibrocartilage which, although it produces a clinical improvement, does not possess the mechanical properties or the composition of hyaline cartilage.

If the damage is too great and in the case of a relapse and/or recurrence, the replacement of all the articular cartilage and his replacement or resurfacing by using metals, plastics materials such as high density polyethylene or ceramic bearing surfaces is tested a success rate which remains limited owing to the poor resilient quality of these substitutes, and to the friction and wear caused by said substitutes on the other articular ends. These substitutes are fixed to the bone by way of bone cement with bone growth stimulation or by way of adhesion using poly(methyl methacrylate) (PMMA) adhesives.

Other less destructive and more reconstructive methods are also implemented and methods involving grafting and implanting autologous chondrocytes have been developed and applied with relative success in young patients.

Autologous cartilage and bone may also be transplanted in the form of pin-shaped portions removed from articular regions of the patient which have been subjected to low levels of stress. Among all the grafting or regrowth stimulation methods, some have now been approved by the health authorities and may be supplemented and encouraged by local administration of growth factors and various products which may limit the risks of rejection and infection.

In most cases, it is difficult to achieve a balance between the need for further grafts and protection of the regions having received grafts and the need to quickly remobilise patients so as to avoid complications caused by an extended period of immobilisation.

Pyrolytic carbon has extremely beneficial mechanical properties for use as an orthopaedic implant.I Its modulus of elasticity is close to that of cortical bone. It has been established through its use as an articular implant in the hand and as a coating for cardiac valves that it is fully biocompatible and does not induce any inflammatory reaction. It also enables good biological fixation and does not induce complications and the cellular growth on pyrolytic carbon is considered to be acceptable. Its modulus of elasticity, which is between 20 and 25 GPa for a density between 1.7 and 2.0 g/cm³ (for bone the respective values are 15 to 20 GPa and 2.0 g/cm³) eliminates the stresses and necrosis observed with metal prostheses.

Pyrolytic carbon is obtained by thermal decomposition of gaseous hydrocarbon at a high temperature by means of a process known as Chemical Vapour Deposition (CVD). In theory, if this process makes it possible to deposit almost all the metal or non-metal elements on numerous supports, graphite has the most beneficial properties for depositing pyrolytic carbon and, more specifically, its thermal expansion coefficient.

In order to produce implants coated with pyrolytic carbon, a graphite substrate is placed in a chamber which is heated to between 1200° C. and 1500° C. then a hydrocarbon gas, such as propane, is added and the extreme temperature destroys the carbon-hydrogen bonds and enables carbon atoms to be deposited on the graphite substrate. Layers between 300 and 600 microns thick are thus deposited on the substrates. The physical and mechanical properties of the material obtained are half-way between those of graphite and those of diamond.

U.S. Pat. No. 4,224,699 discloses a cap-shaped prosthesis to be fixed, without adhesive, to the femoral head by means of an anchoring pin passing through the prosthesis and penetrating the bone. The prosthesis comprises apertures for bone growth or cartilage regeneration. The prosthesis may be made of ceramics or pyrolytic carbon whilst the pin is made of metal or a metal alloy. The drawback of this solution is that it implements two separate materials of which the deformation characteristics are different. Central anchoring by way of a pin may cause unacceptable responses when the joint is moved.

Resurfacing orthopaedic implants are known from US2007/0225822 which are formed of a metal substrate coated with pyrolytic carbon or a pyrolytic carbon alloy. As above, the implant comprises a part for anchoring in the bone.

The objective of the present invention is to provide a new implant which strikes a balance between cartilage reconstruction and rapid remobilisation of the joints.

A further objective of the invention is to provide an implant which is easily arranged in place.

An object of the invention is thus a cartilage resurfacing implant for replacing, shape for shape, all or some of the articular cartilage and optionally part of the subjacent bone, the implant being made of a biocompatible material of which the modulus of elasticity is between 20 and 25 GPa, having an inner surface shaped so as to match the surface of the subjacent bone or cartilage and comprising at least one aperture enabling at least one plug to be arranged and fixed in place.

The invention also relates to a kit comprising an implant of this type and at least one plug.

The invention also relates to a resurfacing process, in which on the one hand an implant of this type and on the other hand at least one plug arranged in the at least one aperture are fixed to the surface of the cartilage or bone by way of a biocompatible adhesive.

Apertures is understood as an opening, through a plenty space, through which light passes.

The invention makes it possible to replace, shape for shape, all or some of the articular cartilage and optionally part of the subjacent bone, i.e. the cartilage and/or part of the bone is replaced after surgical resection or the cartilage and/or part of the bone is replaced, having been previously destroyed by wear or the like. “Shape for shape” means that the shape of the inner surface of the implant, i.e. the surface facing the bone or cartilage, complements and matches, as closely as possible, the shape of the surface on which the implant will be placed. In an embodiment, “shape for shape” means further that the implant has the shape and volume of the cartilage or cartilage plus bone withdrawn and/or to be replaced.

The implant makes it possible to carry out a resurfacing procedure which enables the patient to be quickly remobilised. The present invention makes it possible to reconstruct cartilage by way of resurfacing, whilst also enabling grafts and implants to be used and the cartilage surface to heal as well as rapid remobilisation of the patient whilst protecting the grafted regions.

The apertures formed in the sheet of said material make it possible to arrange the plugs (or studs) in place. These plugs may either simply be placed in the aperture, preferably in contact with the subjacent bone or cartilage, or be anchored in the cavities formed in the regions of cartilage and/or bone. The plugs may serve where bone or cartilage regrowth is to be stimulated and/or as anchoring means for the whole implant.

According to one embodiment of the invention, the inner surface of the implant is shaped so as to be attached to the subjacent bone or cartilage by way of a biocompatible adhesive, e.g. a bone cement.

According to another embodiment of the invention, this attachment is done or reinforced by arranging and fixing in place one or more plugs. The plugs may help to fix the implant in place in accordance with two methods which may be combined. Firstly, the implant comprises a plurality of apertures arranged in such a way that once the plugs have been fixed to the subjacent bone or cartilage, they ensure geometrical locking (means that the plug arrangements makes that when the plugs are fastened to the bone or cartilage, it becomes impossible or difficult to withdraw the implant due to the localization of several apertures and plugs and their differing orientations. For example, the apertures are arranged radially in such a way that it is impossible to remove the implant whilst the plugs are fixed to the bone or cartilage. Secondly, the aperture(s) is/are geometrically shaped in such a way that once the plug(s) has/have been fixed to the subjacent bone or cartilage, the shape is locked in place. The plug is conical for example, the base of the frustum of the cone being in contact with the bone or cartilage.

In one embodiment, said plugs or studs are arranged in such a way that their (outer) surface is in line with the (outer) surface of the implant so the region of articular contact has a perfectly planar surface with no region able to create stress and so the plugs or studs are not subjected to stress when the treated joint is remobilised.

In another embodiment, said plugs or studs are arranged in such a way that their surface is lower than that of the implant so their surface is protected and is not subjected to friction.

In another embodiment, said plugs or studs are arranged in such a way that their surface is in relief, i.e. is higher than that of the implant so they can sustain the stresses at the joint and protect the implant.

These different embodiments correspond to different objectives with regard to joint reconstruction.

These plugs may be cylindrical, conical or parallelepipedal.

Depending on the damage and wear of the articular cartilage, either complete or partial cartilage resection will be carried out and this resection will be replaced with an implant or the cartilage will simply be covered by the implant so as to protect and stimulate its regrowth by arranging said plugs in place.

In one embodiment, the implant according to the invention is in the form of a spherical cap.

In one embodiment, the implant according to the invention is characterised in that the material is made of pyrolytic carbon arranged on a substrate constituting a support, in particular a graphite substrate.

In this embodiment, a graphite substrate is produced in the form of a graphite sheet in the shape of a spherical cap comprising apertures, said substrate then undergoing the process described above so as to obtain an implant having a deposit of pyrolytic carbon.

In this embodiment, the implant may be between 1.5 and 3.5 mm thick.

In one embodiment, the implant according to the invention is characterised in that the material is made of pyrolytic carbon having no substrate constituting the support or only part thereof. For example, once the pyrolytic carbon has been deposited, the implant obtained is processed and cut so as to remove the graphite or the lower layer of pyrolytic carbon and graphite so as to obtain a cap formed solely of a sheet of pyrolytic carbon.

In this embodiment, the implant according to the invention may be between 0.5 and 1.5 mm thick.

In one embodiment, the implant is shaped so as to be implanted on an articular bone head.

In one embodiment, the implant is shaped so as to be implanted in a glenoid cavity.

The implant is fixed on the bone end by way of biocompatible adhesives, such as PMMAs, fibrins or bone cements. The same adhesive is preferably used to adhere the plugs.

The invention also relates to a kit for resurfacing cartilage, comprising at least one implant according to the invention and at least one plug.

According to the invention, the plug may be cylindrical in shape with an annular cross-section for treating cartilage lesions.

In one embodiment, the plug is made of a biomaterial comprising a biological or pharmaceutical active ingredient, in particular selected from the group consisting of growth factors, pharmaceutical active ingredients, and/or tissue extracts, either alone or mixtures thereof.

In one embodiment, the biomaterial is selected from hydrogels, such as polysaccharides, biocompatible polymers, such as polyurethanes, lactic acid polymers (PLLA) or polyhydroxylalkanoates (PHA) in the form of flexible structures, collagen derivatives or mixtures thereof either alone or in combination.

In order to stimulate tissue regeneration, the plugs may comprise growth factors.

The plugs may also comprise active ingredients able to treat or prevent disease or complications and able to keep the implanted site sterile. In one embodiment, the pharmaceutical active ingredients are selected from antiviral agents and antibacterial agents.

Tissue extracts are living biological tissue extracts or autologous, allogeneic or xenogeneic cells. These cells are preferably selected from those able to stimulate regeneration of cartilaginous tissues, for example isolated chondrocytes and multiplied by methods known by the person skilled in the art, such as cell culture from a cartilage biopsy.

In one embodiment, the tissue extracts are selected from autologous, allogeneic or xenogeneic cells belonging to the chondrocyte line or the chondrocyte progenitor cell line.

The invention also relates to a process for resurfacing cartilage so as to replace, shape for shape, all or some of the articular cartilage and optionally part of the subjacent bone, wherein on the one hand an implant made of a biocompatible material of which the modulus of elasticity is between 20 and 25 GPa, which has an inner surface shaped so as to match the surface of the subjacent bone or cartilage and comprises at least one aperture as defined above, and on the other hand at least one plug arranged in at least one aperture are fixed to the surface of the cartilage or bone by way of a biocompatible adhesive.

Depending on the damage and wear of the articular cartilage, the implant and the at least one plug are arranged in place after partial or complete cartilage resection. The plugs may be placed in part in a cavity in the cartilage or bone, either an existing cavity or a cavity made by the surgeon. The plug will help in bone or cartilage growth and recolonisation in particular in an existing cavity.

In another embodiment of the process, the implant and the at least one plug are arranged in place after complete cartilage resection and after resection of the subjacent bone to a specific depth.

According to one embodiment, one or more plugs are arranged in such a way that their surface is in line with the surface of the implant.

According to another embodiment, one or more plugs are arranged in such a way that their surface is lower than the surface of the implant.

According to another embodiment, one or more plugs are arranged in such a way that their surface is higher than the surface of the implant.

In an embodiment, two or three of these three embodiments are combined in the same implant.

According to a feature, the plugs in the apertures participate to the fastening of the implant. A biocompatible adhesive, e.g. a bone cement, is used to attach the plugs and/or the implant itself. In an embodiment, attachment with an adhesive and with the help of the plugs is performed.

The invention will be better understood in light of the embodiments shown in the figures, which are schematic drawings showing non-limiting examples of various embodiments.

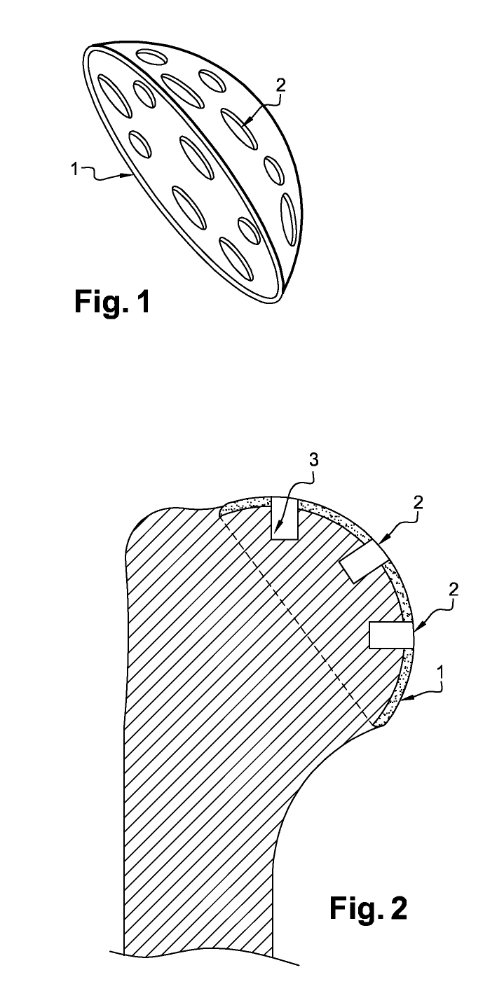

FIG. 1 is a pyrolytic carbon implant according to the invention.

FIG. 2 is a kit according to the invention arranged on an articular end.

The cap shown in FIG. 1 is formed of a sheet of pyrolytic carbon 1 comprising apertures 2. Said apertures 2 are formed, for example, by processing the graphite substrate before it undergoes the process of pyrolytic carbon deposition.

The humeral head shown in FIG. 2 is shown in cross-section so as to illustrate the implant formed of the pyrolytic carbon sheet 1 according to the invention, with the plugs 3 in the apertures 2, i.e. an assembly constituting the kit according to the invention and arranged after implantation. An adhesive may be used to secure the implant and the plugs on and in the bone. The radial arrangement of the apertures and plugs fasten the implant on the humoral head further.

1-27. (canceled)

28. An implant for replacing all or some of an articular cartilage, the implant being made of a biocompatible material of which the modulus of elasticity is between 20 and 25 GPa, the implant having an inner surface shaped to match a surface of subjacent bone or cartilage, the implant comprising at least one aperture through which at least one plug is adapted to be inserted and fixed in place.

29. The implant of claim 28, wherein the implant is a spherical cap.

30. The implant of claim 28, wherein the biocompatible material is pyrolytic carbon.

31. The implant of claim 30, wherein the pyrolytic carbon is arranged on a supporting substrate.

32. The implant of claim 31, wherein the pyrolytic carbon is only partially supported by the supporting substrate.

33. The implant of claim 31, wherein the supporting substrate is made of graphite.

34. The implant of claim 30, wherein the pyrolytic carbon has no supporting substrate.

35. The implant of claim 28, wherein the implant is between 1.5 and 3.5 mm thick.

36. The implant of claim 30, wherein the implant is between 0.5 and 1.5 mm thick.

37. The implant of claim 34, wherein the implant is between 0.5 and 1.5 mm thick.

38. The implant of claim 28, wherein the inner surface is shaped to be attached to the subjacent bone or cartilage with a biocompatible adhesive.

39. The implant of claim 38, wherein the one or more plugs is configured to reinforce attachment of the inner surface to the subjacent bone or cartilage.

40. The implant of claim 28, wherein the at least one aperture is a plurality of apertures, wherein the at least one plug is a plurality of plugs, the plurality of apertures having an arrangement that causes geometrical locking when the plurality of plugs is inserted through the plurality of apertures and fixed into place.

41. The implant of claim 28, wherein the at least one aperture is geometrically shaped so as to lock the implant in place when the at least one plug is inserted therethrough and fixed to the subjacent bone or cartilage.

42. The implant of claim 28, wherein the at least one aperture is formed in a shape selected from the group consisting of a cylinder, a cone, and a parallelepiped.

43. A kit for replacing all or some of an articular cartilage, the kit comprising:
an implant made of a biocompatible material of which the modulus of elasticity is between 20 and 25 GPa, the implant having an inner surface shaped to match a surface of subjacent bone or cartilage, the implant comprising at least one aperture; and
at least one plug adapted to be inserted through the at least one aperture and fixed in the subjacent bone or cartilage.

44. The kit of claim 43, wherein a shape of the at least one plug is selected from the group consisting of a cylinder, a cone, and a parallelepiped.

45. The kit of claim 43, wherein the at least one plug is made of a biomaterial.

46. The kit of claim 45, wherein the biomaterial comprises an active ingredient, or a mixture of two or more active ingredients, selected from the group consisting of growth factors, pharmaceutical active ingredients, and tissue extracts.

47. The kit of claim 45, wherein the biomaterial is comprised of one or more biocompatible hydrogels and polymers.

48. The kit of claim 47, wherein the biomaterial is a material, or a mixture of two or more materials, selected from the group consisting of: hydrogels made of polysaccharides and polyurethanes, lactic acid polymers (PLLA) or polyhydroxylalkanoates (PHA) in the form of flexible structures, and collagen derivatives.

49. The kit of claim 45, wherein the biomaterial comprises one or more of an antiviral agent and an antibacterial agent.

50. The kit of claim 45, wherein the biomaterial comprises autologous, allogeneic, or xenogeneic cells belonging to the chondrocyte line or the chondrocyte progenitor line.

51. A process for cartilage resurfacing for replacing all or some of an articular cartilage, the process comprising:
affixing an implant to a surface of subjacent bone or cartilage with a biocompatible adhesive, the implant being made of a biocompatible material of which the modulus of elasticity is between 20 and 25 GPa, the implant having an inner surface shaped to match the surface of the subjacent bone or cartilage, the implant comprising at least one aperture;
inserting at least one plug through the at least one aperture; and
affixing the at least one plug to the subjacent bone or cartilage with the biocompatible adhesive.

52. The process of claim 51, further comprising:
at least partially resecting the cartilage prior to affixing the implant and the at least one plug.

53. The process of claim 52, further comprising:
resecting the subjacent bone to a specific depth prior to affixing the implant and the at least one plug.

54. The process of claim 51, further comprising aligning an outer surface of the one or more plugs with an outer surface of the implant.

55. The process of claim 51, further comprising placing an outer surface of the one or more plugs closer to the subjacent bone or cartilage than an outer surface of the implant.

56. The process of claim 51, further comprising placing the one or more plugs such that an outer surface of the one or more plugs protrudes beyond an outer surface of the implant.
